(19) United States
(12) Patent Application Publication
(10) Pub. No.: US 2021/0259285 A1
(43) Pub. Date: Aug. 26, 2021

(54) USE OF A COMPOSITION COMPRISING MARINE OIL AND JUICE FOR IMPROVING MUSCLE PERFORMANCE

(71) Applicant: SMARTFISH AS, Oslo (NO)

(72) Inventor: Janne Sande Mathisen, Oslo (NO)

(21) Appl. No.: 17/197,765

(22) Filed: Mar. 10, 2021

Related U.S. Application Data
(62) Division of application No. 16/304,896, filed on Nov. 27, 2018, now abandoned.

(30) Foreign Application Priority Data
May 27, 2016 (NO) 20160913

(51) Int. Cl. A23L 2/06; A23L 29/231; A23L 33/105; A23L 33/115; A23L 33/125; A23L 2/56; A23L 2/60; A23L 2/02; A23L 2/52

(52) U.S. Cl. CPC A23L 2/06; A23V 2250/5484; A23L 33/105; A23L 33/115; A23L 33/125; A23L 2/56; A23L 2/60; A23L 2/02; A23L 2/52; A23V 2002/00; A23V 2200/316; A23V 2250/1868; A23V 2250/187; A23V 2250/54252; A23L 29/231

(57) ABSTRACT

A composition comprising a combination of marine oil and juice in oil-in-water emulsion, for improving muscle performance.

### FIELD OF THE INVENTION

The present invention provides a composition comprising marine oil and juice in an oil-in-water emulsion for improving muscle performance.

### BACKGROUND OF THE INVENTION

Over the last years, there has been extensive research conducted on the role of nutritional compositions and daily supplementation or during exercise to enhance endurance and exercise performance following exercise. The muscle recovery time is aimed to be reduced as well.

Substrate availability to skeletal muscle is paramount for optimizing endurance performance. During endurance exercise, an increase in carbohydrate (CHO) oxidation has been shown to help reduce fatigue and increase performance (Jeukendrup, 2010). Interestingly, in the last two decades, omega-3 polyunsaturated fatty acids (n-3 PUFA), in particular, the active ingredients eicosapentaenoic acid (EPA) and docosahexaenoic acid (DHA) have been proposed to help increase CHO oxidation, by increasing the ability of muscle cells to utilize glucose through their unsaturation actions on the skeletal muscle membrane (Andersson et al., 2000, Vessby et al., 2002). In addition, supplementation with n-3 PUFAs has further been linked to a reduction in oxygen (O2) consumption and observed in both animal (Pepe and McLennan, 2002, 2007) and human (Peoples et al, 2008) models. This reduced oxygen cost, and hence improved oxygen efficiency, may be attributed to a shift in the fuel source predominantly utilized by skeletal muscle during exercise.

Omega-3 polyunsaturated fatty acids (n-3 PUFA) can mediate numerous biological processes. Given their action in changing skeletal muscle membrane integrity, n-3 PUFA supplementation may increase glucose availability, thereby influencing fuel metabolism at rest and/or during exercise.

WO 2007/064222 discloses a composition comprising low oxidized fish oil and juice in an oil-in-water emulsion. There is however, no disclosure of any biological effect in relation to improved muscle performance and/or extended endurance during exercise including improved muscle recovery.

Thus, composition that improves muscle performance is desired.

### SUMMARY OF THE INVENTION

The present invention provides a composition comprising a combination of marine oil and juice in an oil-in-water emulsion, for improving muscle performance wherein said marine oil is selected from marine oil having a totox value below 20 and omega-3 content above 10% by weight based on the total weight of the fish oil and wherein a suitable emulsifier is used to stabilize the emulsion.

The composition of the invention is beneficial for improved muscle performance, including the following conditions: extended endurance during exercise; reduced “delayed onset of muscle soreness (DOMS)”; reduced acute muscle soreness; reduced muscle recovery time; reduced exercise-induced muscle damage; and increased exercise capacity.

Preferred embodiments are set forth in the dependent claims and in the detailed description of the present invention.

### DESCRIPTION OF THE FIGURES

The following abbreviations are used:

FO: juice-based Fish Oil composition according to the invention
PRO: protein containing control drink
CHO: carbohydrate containing isocaloric drink
CON: taste-matched control drink containing no fish oil

FIG. 1 illustrates a schematic diagram of experimental protocol of Example 1.

Measurements of muscle soreness, anti-inflammatory markers, and muscle function were collected before and during the recovery period (24, 48 and 72 h).

FIG. 2 illustrates the percentage of n-3 PUFA/Total PUFA composition in blood before and after 6 weeks of supplementation. Data are expressed as means±SEM.

* Significantly different from Pre.

FIG. 3 illustrates the general muscle soreness measured as VAS score (visual analoge score) of the dominant leg prior to, and during 72 h recovery periods from EIMD (Exercise-Induced Muscle Damage). *FO significantly different from PRO and CHO. ** FO significantly different from PRO.

FIG. 4 illustrates results of a soccer-specific test, i.e. yoyo-test during 72 h recovery period.

FIG. 5 illustrates the percentage change in serum creatine kinase (CK) concentration. * Significantly different from FO. # Tendency (p=0.07) to be different from FO.

FIG. 6 illustrates percentage change in serum c-reactive protein (CRP) concentration prior to, and during 72 h recovery period from EIMD.

FIG. 7 illustrates a schematic representation of experimental protocol of Example 2. Measurements of indirect calorimetry before and after a 4 weeks supplementation of either a composition of the invention or a control supplement.

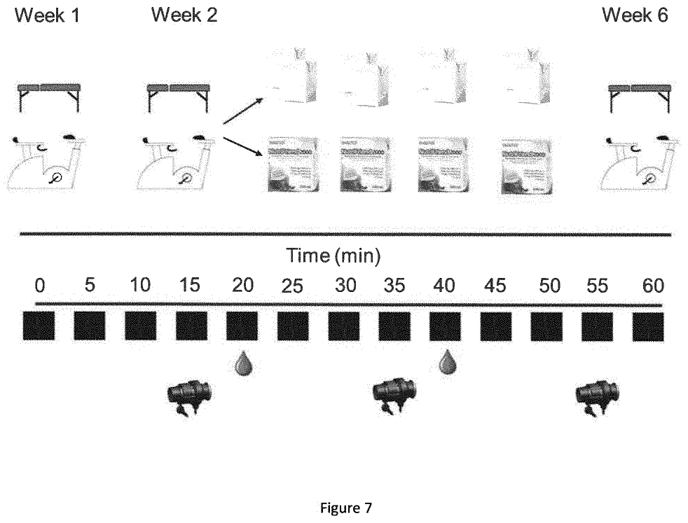

FIG. 8 illustrates percentage of n-3 PUFA/Total PUFA composition in blood before and after 4 weeks supplementation. Data expressed as means±SD.

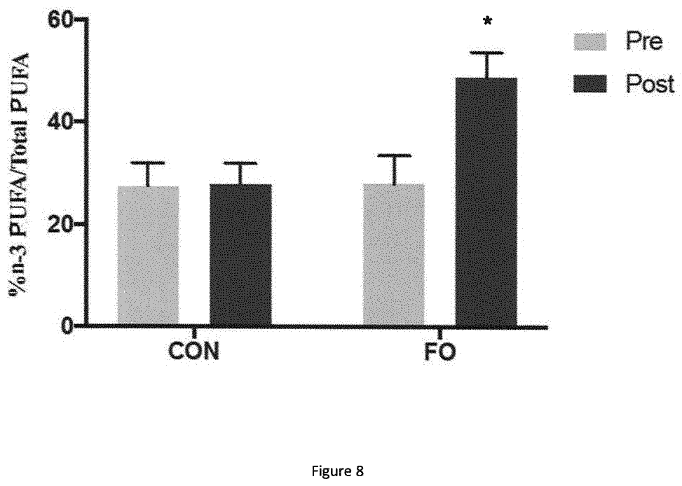

* Significantly different from Pre.

FIG. 9 illustrates respiratory Exchange Ratio during exercise Pre and Post supplementation. *Significant difference in Pre to Post changes between the composition of the invention group (FO) and Control group (CON). Values are presented as means±SD.

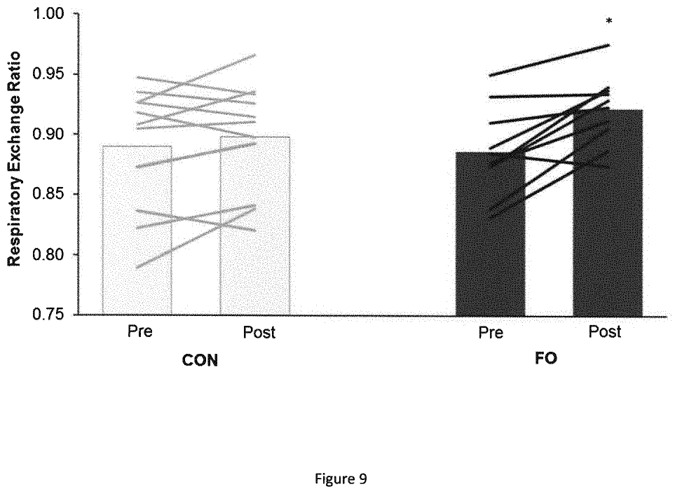

FIG. 10 illustrates the carbohydrate (CHO) oxidation rates Pre and Post supplementation. *Significant difference in Pre to Post changes between the composition of the invention group (FO) and Control group (CON) (p<0.05). Values are presented as mean±SD.

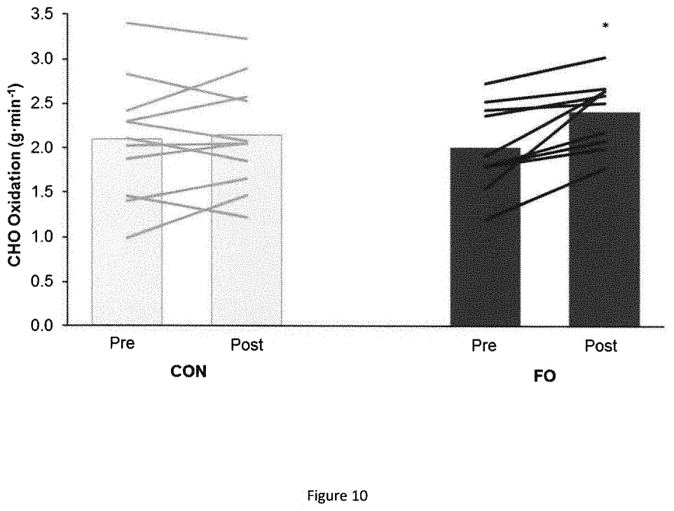

FIG. 11 illustrates the fat oxidation rates during exercise Pre and Post supplementation. *Significant difference in Pre to Post changes between the composition of the invention group (FO) and Control group (CON) (p<0.05). Values are presented as mean±SD.

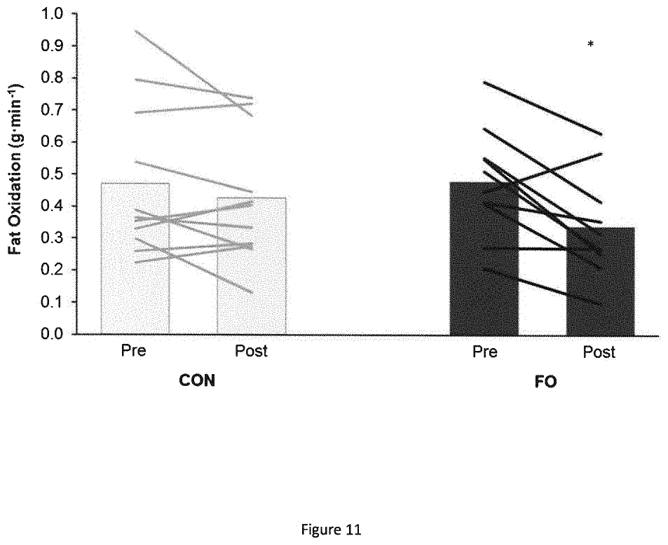

### DETAILED DESCRIPTION OF THE INVENTION

It is an object of the present invention to provide a composition of the present invention for improving muscle performance.

Another object of the present invention to provide a composition that will extend endurance during exercise.

Another object of the present invention to provide a composition that will reduce “delayed onset of muscle soreness (DOMS)”.

Another object of the present invention to provide a composition that will reduce acute muscle soreness.

Another object of the present invention to provide a composition that will reduce muscle recovery time.

Another object of the present invention to provide a composition that will reduce exercise-induced muscle damage.

Another object of the present invention to provide a composition that will increase exercise capacity.

Another object of the present invention to provide a composition that will increase the carbohydrate (CHO) oxidation to help reduce fatigue and increase performance.

It has now surprisingly been found that the composition of the invention is useful as a nutritional supplement revealing improved muscle performance.

The composition used in the present invention has been shown to have high stability both chemically and physically. The composition enables low oxidation and keeps the vulnerable nutrients intact and potent which in turn results in increased absorption and high bioavailability.

The composition comprises:
a) marine oil with a low totox value, especially omega-3 fatty acids in concentrations sufficient to achieve nutritional effect(s) and
b) juice containing naturally present antioxidants;
c) emulsifier(s)

Different aspects of the composition are described in WO 2007/064222, which is the inventor's own patent, although further modifications of the composition may be employed in the present invention.

Marine oil in a composition often contributes to an unwanted fishy taste and after taste. The composition for use in the present invention has the advantage of e.g. being a tasteful drink which may be an important prerequisite in patients experiencing changes is in taste and loss of appetite.

Accordingly one aspect of the present invention relates to a composition comprising a combination of marine oil and juice in an oil-in-water emulsion for improving muscle performance, wherein said marine oil is selected from marine oil having a totox value below 20 and omega-3 content above 10% by weight based on the total weight of the marine oil, and wherein a suitable emulsifier is used to stabilize the emulsion.

Use of the composition of the invention may be beneficial to adverse effects on muscles induced by moderate to strenuous exercise.

Thus, the present invention relates to a composition comprising a combination of marine oil and juice in oil-in-water emulsion, for improving muscle performance, wherein said marine oil is selected from marine oil having a totox value below 20 and omega-3 content above 8% by weight based on the total weight of the marine oil and wherein a suitable emulsifier is used to stabilize the emulsion.

All aspects of improved muscle performance are included within the scope of the invention, in particular conditions selected from the group comprising: extended endurance during exercise; reduced “delayed onset of muscle soreness (DOMS)”; reduced acute muscle soreness; reduced muscle recovery time; reduced exercise-induced muscle damage; and increased exercise capacity.

The composition of the invention provides improved muscle performance by facilitated muscle recovery from damaging exercise. A further beneficial effect of the present composition is a reduction of the incidence of injuries and strain as the muscles are better protected leading to less severe muscle damage and soreness. Further, the composition of the invention provides improved muscle performance by increasing the carbohydrate (CHO) oxidation to help reduce fatigue and increase performance. Further, the composition of the invention provides improved muscle performance by facilitating muscle explosively.

In this context, marine oil used in the composition are any marine oil prepared from fresh marine species where all process steps are conducted carefully and under strict oxygen control according to functional oil standards in order to prevent oil oxidation. The marine oil will have a low oxidative status, revealing colourless oil without the characteristic smell or taste of e.g. fish. The level of oxidation given as the totox value (2 times the peroxide value (PV) added with the anisidin value (AV)) should be below 20, preferably below 15, more preferably below 10, and most preferably below 5. Marine oil present in many food supplements today contains oil with a much higher totox value, typically 20-30 or even higher.

Thus, in one preferred embodiment of the present invention, the marine oil of the composition is having a totox value below 20, preferably below 15, more preferably below 10, and most preferably below 5.

Marine oils are rich in omega-3 fatty acids. However, the content is varying. In one preferred embodiment of the present invention, the content of omega-3 fatty acids in the marine oil should be at least 8%, preferably at least 12%, more preferably at least 16%, most preferably about 20-35% based on the weight of the marine oil.

According to the invention the marine oil may be any oil rich in omega-3 fatty acids, e.g. fish oil, seal oil or krill oil. The marine oil may be mixed with other polyunsaturated oils of vegetable origin such as algae oil and herbal oil such as evening primrose oil and rapeseed oil.

In one preferred embodiment of the present invention the composition of the combination comprises marine oil in an amount of about 0.5% to about 15% by weight based on the total weight of the marine oil formulation, preferably in the range of about 0.5% to about 10%, more preferably in the range of about 0.5% to about 7%, most preferably in the range of about 1.5% to about 6%, even more preferably in the range of about 5%-6%.

The composition comprises marine oil in an oil-in-water emulsion wherein the water phase comprises juice containing natural antioxidants. The juice may be selected from fruit, berry or vegetable juices, or any combination thereof. Preferably, the juice is obtained from fruit, berry or vegetables having a suitable high level of antioxidants. Further, it is preferred that the juice possess a minimum level of metal ions functioning as oxidizing agents. Preferred juices may be selected from the group of pomegranate, apricot, grapefruit, orange, cranberry, rosehips, pineapple, black chokeberry, aronia, mulberry, cloudberry, acerola, raspberry, watermelon, peach, grape, cherry, carrot, apple, pear, lemon, beetroot, passion fruit, mango, beet root, carrot and kiwi.

The juice may be fresh pressed juice or juice in the form of juice concentrate. The juice concentrate is diluted to obtain a normal ready-to-use juice prior to formulation. Preferred juice or juice concentrates are those selected from the group consisting of apple, pear, pomegranate, chokeberry/aronia, passion fruit and combinations thereof.

The content of juice is about 20%-97% by weight based on the total weight of the composition. When juice concentrate is used, 20-97% refers to the amount of juice diluted to obtain a normal ready-to-use juice. In a preferred embodiment the content of juice is 30-97%, preferably 50%-95%, more preferable 75%-95% and most preferable 80%-95%. The water phase is further balanced with water if applicable.

In the preparation of the composition any suitable emulsifier or combinations of emulsifiers, may be used. Milk solids, whey protein and pectin, pea proteins, fish proteins, hemp proteins, soya proteins or any combination thereof are preferred emulsifiers. Whey protein is preferred, in particular in the form of whey protein powder or whey protein isolate.

The composition may further comprise additional proteins and amino acids, added as a source of nutrition. Example of such is citrulline.

Addition of creatine is also encompassed.

The composition may further comprise sweetener, flavouring agents and preservatives. As such the composition may be given any desirable taste.

A suitable preservative is potassium sorbate. However any suitable preservative may be employed. A suitable sweetener is xylitol. However any suitable sweetener may be employed.

Suitable further flavouring agents are jackfruit and rosemary extract. Additionally, pH regulating agents such as ascorbic acid may be added.

The composition may also include one or more additionally added antioxidants, including but not limited to, tocopherols, curcumin, resveratrol, polyphenols, carotenoids, coenzyme Q10, lycopene and lutein.

The composition may also include one or more vitamins such as, for example, vitamin B, C and/or D. Further, the composition may also include one or more minerals such as, for example, magnesium, potassium and/or sodium.

The composition may also comprise yoghurt powder. By adding yoghurt powder, the formulation thickens giving an inviting consistency. The amount of yoghurt powder may be in the range of 5-10% by weight of the composition. In another embodiment, the composition may be added prebiotics and/or probiotics. In yet another embodiment, the composition may be carbonated.

The consistency of the composition may vary from a liquid drinkable consistence to a more viscous drink, e.g. a smoothie. Further, the consistency of the composition may be creamy like yoghurt.

Preferably the composition is a drink formulation, although it is also contemplated that the composition may be presented in other well-known administrative forms, such as a tablet or capsule or gel. For example, a drink formulation may be prepared and then dried, e.g. lyophilized to a powder or granulation, and then presented in a tablet or capsule dosage form. Alternatively, a drink formulation could be concentrated to form a concentrate for use by a patient or gelled using standard gelling techniques to provide an edible gel.

Preferably the composition is administered on a daily basis, and most preferably it is a drink formulation administered on a daily basis. As used herein daily basis means administering at least once a day, but also includes multiple administrations in a day, e.g. twice or thrice daily.

Preferably the composition is filled in dose units, preferably airtight bottles or small sealed containers, but any suitable container may be applicable. Containers containing different amounts of fatty acids adjusted to the recommended daily dose of adults, children and infant and number of administration in a day are possible.

The range of each of DHA and EPA in the composition that is administered may be from about 200 mg to about 5000 mg per day, preferably about 3000 mg per day, more preferably about 2000 mg per day and most preferably about 1100 mg or 1000 per day. In the case the formulation according to the invention is provided as a drink formulation, a suitable unit volume is 50-200 ml. In the case the composition of the present invention is provided as yoghurt, a suitable unit volume is 25-100 ml.

Different aspects of the composition are described in WO 2007/064222, although further modifications of the composition may be employed in the present invention.

### EXAMPLES

### Example 1

Improved Exercise Recovery Following Daily Supplementation with a Composition of the Invention.

### Introduction:

Soccer players often experience eccentric exercise-induced muscle damage. An improved muscle functional recovery from eccentric exercise has been shown with protein feeding. Since fish oil derived n-3 polyunsaturated fatty acids (n-3PUFA) exhibit anti-inflammatory properties, combining fish oil with protein may facilitate recovery.

### Purpose:

The purpose of the study was to assess the influence of 6 weeks supplementation with a juice-based antioxidant-containing drink according to the invention containing a combination of n-3PUFA, whey protein and vitamin D on muscle function and soreness, inflammation and soccer performance during exercise recovery.

### Methods:

Soccer players (n=30) were assigned to one of three conditions. The experimental condition identified FO contained n-3PUFA (550 mg DHA, 550 mg EPA), whey protein (15 g), carbohydrate (14 g) and vitamin D (3 μg) and is a drink according to the invention available from Smartfish AS identified Recharge High Protein. The protein control condition (PRO) contained whey protein (15 g), carbohydrate (14 g) and vitamin D (3.9 μg). The eucaloric control condition (CHO) contained carbohydrate. The nutritional compositions of the supplements are set forth in Table 1 below.

TABLE 1
The nutritional compositions of the supplements.
Each supplement was taken twice a day for 6 weeks

| | FO | PRO | CHO |
|---|---|---|---|
| Volume (ml) | 200 | 200 | 200 |
| Energy Value (kcal) | 200 | 150 | 200 |
| EPA (mg) | 550 | — | — |
| DHA (mg) | 550 | — | — |
| Vitamin D (μg) | 3 | 4 | — |
| Whey Protein (g) | 15 | 15 | — |
| Carbohydrate (g) | 14 | 14 | 24 |

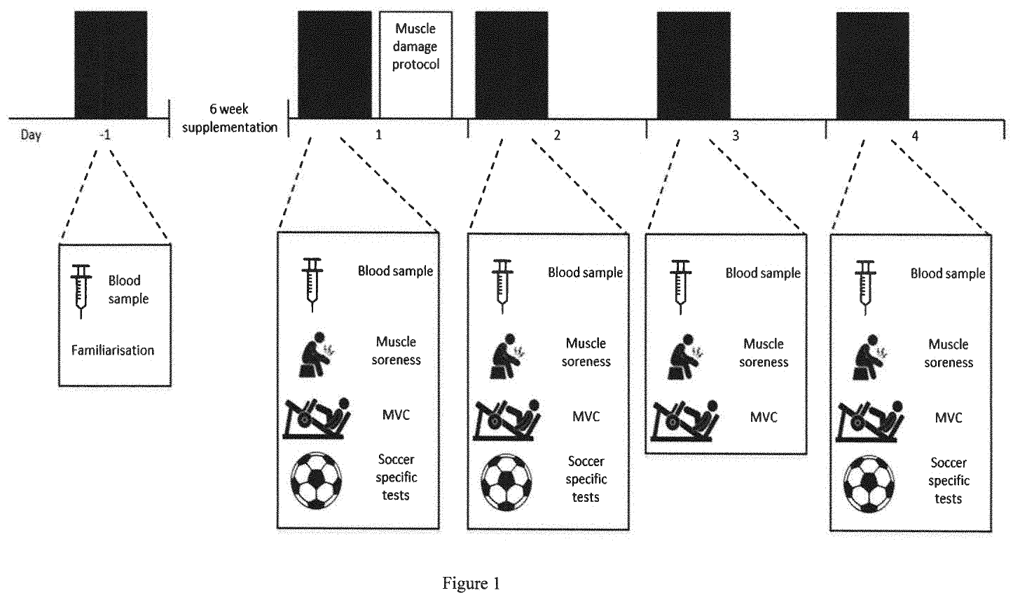

The study design is illustrated in FIG. 1.

Soccer specific test well known in the art such as CMJ (Counter Movement Jump), LSPT (Loughborough Soccer Passing Test) and Yoyo test, were performed. Eccentric exercise consisted of 12 sets unilateral knee extension/flexion contractions on both legs separately. Statistical analyses included conventional p-value and magnitude-based inferential approaches.

### Results:

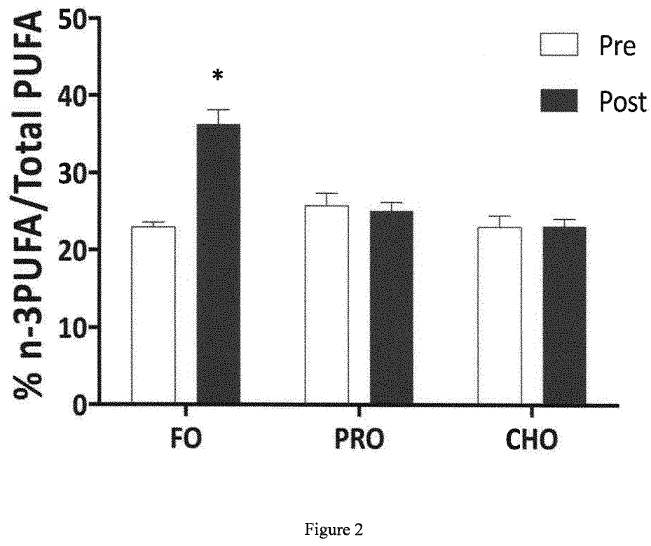

Post supplementation, blood % n-3PUFA/total PUFA was greater in FO (36±2) vs. PRO (25±1, p<0.05) or CHO (23±1, p<0.05) as set forth in FIG. 2.

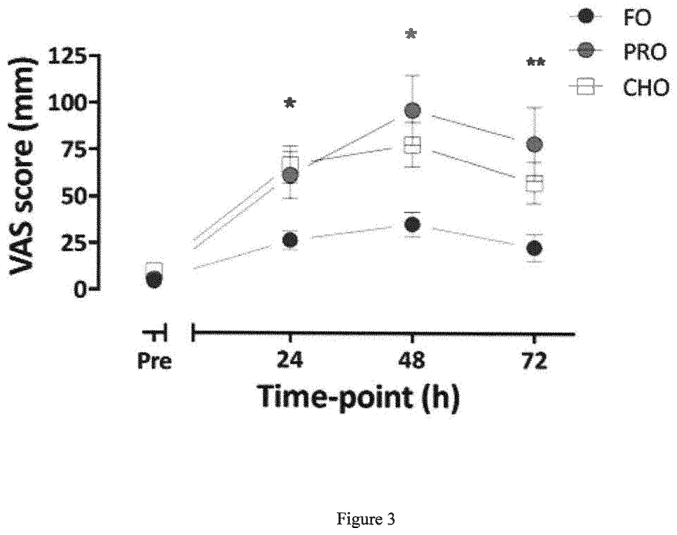

Muscle soreness (non-dominant leg) during 72 h exercise recovery from EIMD (Exercise-Induced Muscle Damage) was significantly less in FO vs. PRO and CHO as set forth in FIG. 3. Muscle soreness expressed as AUC was (1947±364 mm×72 h) in FO vs. PRO (4640±885 mm×72 h, p<0.05) and CHO (4495±618 mm×72 h p<0.0.5).

The results of the soccer specific tests are set forth in table 2 below:

TABLE 2
Magnitude based inferential statistical
analysis of soccer specific tests

| Soccer Specific Test | Groups | Magnitude Based Inferential Statistics |
|---|---|---|
| CMJ | FO vs PRO | 82% likely beneficial effect |
| | FO vs CHO | 85% likely beneficial effect |
| LSPT | FO vs PRO | 78% likely beneficial effect |
| | FO vs CHO | 92% likely beneficial effect |
| Yoyo Score | FO vs PRO | 70% possibly beneficial effect |
| | FO vs CHO | 79% likely beneficial effect |

CMJ; Counter movement jump.
LSPT; Loughborough Soccer Passing Test

FO elicited a possibly (82-85%) beneficial effect on counter movement jump (CMJ) performance vs. PRO and CHO.

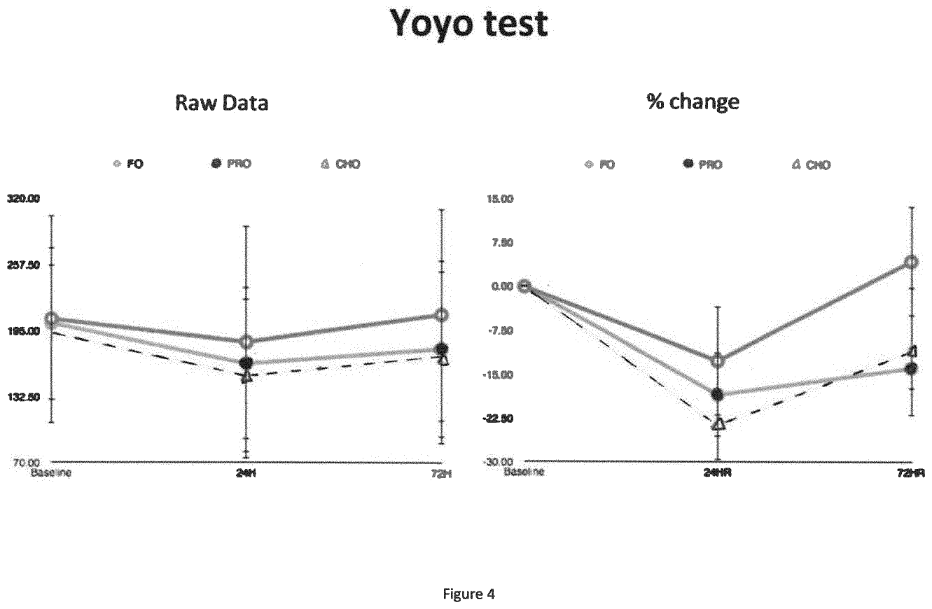

The yoyo test performance during 72 h exercise recovery showed an improved performance in the FO vs PRO and CHO both at 24 h and 72 h as set forth in FIG. 4.

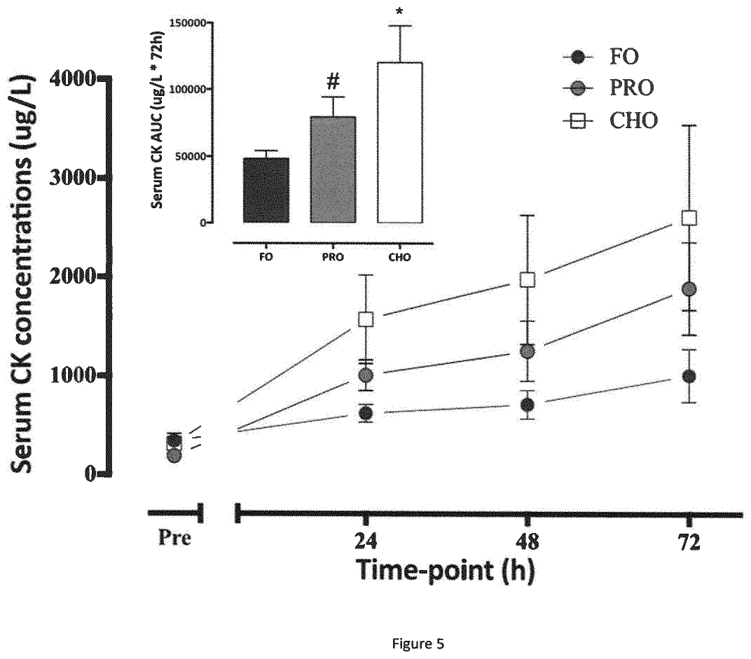

Blood concentrations of creatine kinase were significantly lower in FO vs CHO (p<0.05) and tended to be lower in FO vs PRO (p=0.07) as set forth in FIG. 5. Expressed as AUC the blood concentrations of creatine kinase was less in FO (AUC: 42±11 mg/L×72 h) vs. CHO (AUC: 83±42 mg/L×72 h, p<0.05) and tended (p=0.07) to be lower in FO vs. PRO.

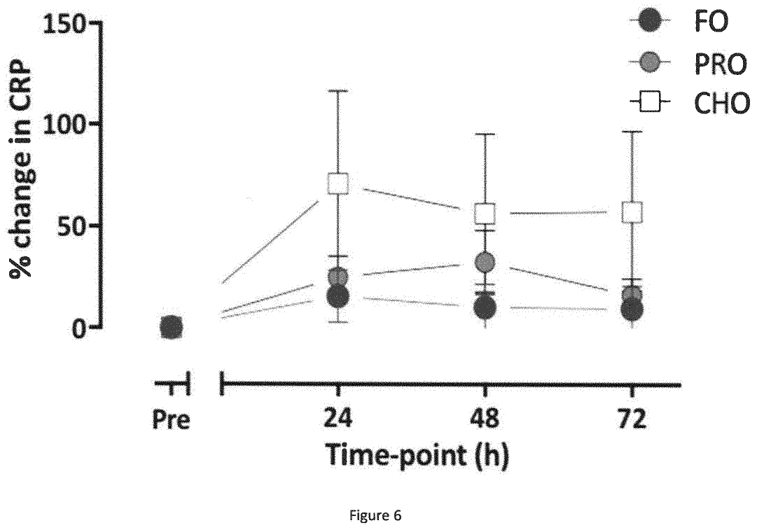

The blood concentration of C-reactive protein was lower in FO vs CHO and tended to is be lower also in FO vs. PRO as set forth in FIG. 6.

### Findings:

It was surprisingly found that 6 weeks supplementation with a juice-based fish oil drink according to the invention, improved muscle function and soreness, reduced inflammation markers and increased soccer performance during exercise recovery compared to a protein control with no fish oil and an isocaloric carbohydrate rich control.

### Example 2

Increase in Carbohydrate (CHO) Oxidation Following Daily Supplementation with a Composition of the Invention.

### Introduction:

Omega-3 polyunsaturated fatty acids (n-3 PUFA) can mediate numerous biological processes, including changing skeletal muscle membrane composition. Given the metabolic role of skeletal muscle, n-3 PUFA supplementation may influence fuel metabolism at rest and/or during exercise.

### Purpose

The purpose of the study was to assess the effects of 4 weeks supplementation with a juice-based drink according to the invention containing a combination of n-3PUFA, whey protein and vitamin D both at rest and during steady-state exercise in endurance-trained individuals.

### Methods

Twenty-one male cyclists and triathletes underwent two experimental trials separated by four weeks. During this period participants were supplemented twice daily (2×200 ml) with a juice based drink according to the invention (FO) containing fish oil, or a taste-matched control drink (CON) containing no fish oil. One unit (200 ml) FO contains 1 g EPA and 1 g DHA and is a drink according to the invention available from Smartfish AS identified Recharge Lipid+.

During the experimental trials, expired gas was collected at rest and during 60 minutes of cycling at 85% of the individual lactate threshold.

### Results

Table 3 and 4 below show the participants' characteristics in both the FO and CON groups prior to and following 4 weeks of supplementation.

TABLE 3
Participant characteristics

| | Comp of Invention (FO) | Control (CON) |
|---|---|---|
| Age (years) | 42 ± 8 | 38 ± 10 |
| Height (cm) | 184.3 ± 5.9 | 181.6 ± 7.4 |
| Body mass (kg) | 80.4 ± 11.2 | 82.7 ± 12.8 |
| Cycling per week (h) | 6.5 ± 1.9 | 6.2 ± 1.9 |
| VO₂ₘₐₓ (ml · kg⁻¹ · min⁻¹) | 59.4 ± 7.1 | 61.0 ± 6.1 |

TABLE 4
Body mass, skinfold measurements and habitual dietary
intake of the participants in both the FO and
CON groups. Data expressed as means ± SD.

| | Comp of Invention (FO) | Control (CON) |
|---|---|---|
| Body mass (Pre) (kg) | 80.4 ± 11.2 | 82.7 ± 12.8 |
| Body mass (Post) (kg) | 81.7 ± 11.5 * | 83.2 ± 12.8 |
| Sum of skinfolds (Pre) (mm) | 65.8 ± 30.4 | 80.0 ± 20.5 |
| Sum of skinfolds (Post) (mm) | 69.5 ± 28.7 | 79.8 ± 19.9 |
| Energy intake (kcal · d⁻¹) | 2816 ± 761 | 2668 ± 811 |
| CHO intake (g · d⁻¹) | 360 ± 134 | 321 ± 101 |
| Protein intake (g · d⁻¹) | 87 ± 25 | 110 ± 32 |
| Fat intake (g · d⁻¹) | 97 ± 37 | 108 ± 30 |

* Significantly different from Pre.

Supplementation with a composition of the invention (FO) significantly increased RER when compared to the control (Δ+0.04±0.03 compared to Δ+0.01±0.02, p<0.05) and also increased CHO oxidation (Δ+0.41±0.32 g·min−1 compared to Δ+0.05±0.30 g·min−1 p<0.05) during steady-state exercise. There also was a reduction in fat oxidation during exercise following supplementation with a composition of the invention (p<0.05).

### Findings:

It was surprisingly found that 4 weeks supplementation with a juice-based drink according to the invention may potentiate a shift in fuel utilization to carbohydrate oxidation during steady-state exercise in endurance-trained cyclists.

### Example 3

Drinks according to the invention comprising citrulline

| | % by weight | kg |
|---|---|---|
| Rosemary extract 201 | 0.0200 | 0.20 |
| Toco 50 | 0.010 | 0.10 |
| Pektin | 0.700 | 7.00 |
| Whey protein powder | 6.000 | 60.00 |
| Citrulline | 1.500 | 15.00 |
| Apple concentrate | 7.000 | 70.00 |
| Pomme granat concentrate | 2.400 | 24.00 |
| Aronia concentrate | 0.800 | 8.00 |
| Passion fruit concentrate | 0.320 | 3.20 |
| Pear concentrate | 4.100 | 41.00 |
| Water, purified deionized | 73.972 | 739.72 |
| Lychee flavouring | 0.008 | 0.08 |
| Jackfruit flavouring | 0.170 | 1.70 |
| Fish oil | 3.000 | 30.00 |
| Vitamin D3* | | |
| | 100.000 | 1000.00 |
| Rosemary extract 201 | 0.0200 | 0.20 |
| Toco 50 | 0.010 | 0.10 |
| Pektin | 0.700 | 7.00 |
| Whey protein powder | 6.000 | 60.00 |
| Coenzym Q10 | 0.150 | 1.50 |
| Citrulline | 1.500 | 15.00 |
| Apple concentrate | 7.000 | 70.00 |
| Pomme granat concentrate | 2.400 | 24.00 |
| Aronia concentrate | 0.800 | 8.00 |
| Passion fruit concentrate | 0.320 | 3.20 |
| Pear concentrate | 4.100 | 41.00 |
| Water, purified deionized | 73.822 | 738.22 |
| Lychee flavouring | 0.008 | 0.08 |
| Jackfruit flavouring | 0.170 | 1.70 |
| Fish oil | 3.000 | 30.00 |
| Vitamin D3* | | |
| | 100.000 | 1000.00 |

### Example 4

Drink according to the invention comprising creatine

| | % by weight | kg |
|---|---|---|
| Rosemary extract 201 | 0.020 | 0.20 |
| Toco 50 | 0.010 | 0.10 |
| Creatin Monohydrat | 1.300 | 13.00 |
| Pektin | 0.600 | 6.00 |
| Whey protein powder | 1.750 | 17.50 |
| Apple concentrate | 7.000 | 70.00 |
| Pomme granat concentrate | 2.400 | 24.00 |
| Aronia concentrate | 0.800 | 8.00 |
| Passion fruit concentrate | 0.320 | 3.20 |
| Pear concentrate | 4.100 | 41.00 |
| Water, purified deionized | 79.922 | 799.22 |
| Vitamin D3 * | | |
| Lychee flavouring | 0.008 | 0.08 |
| Jackfruit flavouring | 0.170 | 1.70 |
| Salmon oil | 1.600 | 16.00 |
| Sum | 100.000 | 1000.00 |
| Rosemary extract 201 | 0.020 | 0.20 |
| Toco 50 | 0.010 | 0.10 |
| Creatin Monohydrat | 1.300 | 13.00 |
| Calcium | 0.150 | 1.50 |
| Pektin | 0.600 | 6.00 |
| Whey protein powder | 1.750 | 17.50 |
| Apple concentrate | 7.000 | 70.00 |
| Pomme granat concentrate | 2.400 | 24.00 |
| Aronia concentrate | 0.800 | 8.00 |
| Passion fruit concentrate | 0.320 | 3.20 |
| Pear concentrate | 4.100 | 41.00 |
| Water, purified deionized | 79.772 | 797.72 |
| Vitamin D3* | | |
| Lychee flavouring | 0.008 | 0.08 |
| Jackfruit flavouring | 0.170 | 1.70 |
| Salmon oil | 1.600 | 16.00 |
| Sum | 100.000 | 1000.00 |

*Each unit volume of 200 ml contains 500 IU of Vitamin D3

The marine oil used in all preparations was Denomga arctic oil from Orkla. The totox value was below 8 and the preparation was conducted according to functional oil standards.

The drinks of Example 3 and 4 were prepared as follows:

### 1. Oil Phase

The oil is mixed with Toco 50 (an antioxidant preparation favourable to the stabilization of the oil). Any further oil soluble ingredients are mixed with the oil. It is important that the oil is protected against oxidation during processing.

### 2. Water Phase

A tank is filled with purified and deionised water. Any water soluble ingredients are added. Thereafter the oil phase is introduced into the water phase after which the emulsifier is added to obtain an emulsion.

The obtained emulsion may alternatively be subjected to a quick pasteurization (about 90° C. for 8 s), followed by homogenization and cooling to a temperature of 4-8° C.

Finally, the drink is filled on airtight aseptic containers, preferably single dose containers, e.g Tetra Brick about 200 ml and stored at 6-8° C. until use.

Strict oxygen control must be implemented in all steps to avoid oxidation of marine oil.

1. A method for improving muscle performance, the method comprising:
administering to a human a composition comprising a combination of marine oil and juice in oil-in-water emulsion to improve muscle performance,
wherein said marine oil is selected from marine oil having a totox value below 20 and omega-3 content above 8% by weight based on the total weight of the marine oil, and
wherein an emulsifier is used to stabilize the emulsion.

2. The method of claim 1, wherein said improved muscle performance is selected from the group consisting of: extended endurance during exercise; reduced delayed onset of muscle soreness (DOMS); reduced acute muscle soreness; reduced muscle recovery time; reduced exercise-induced muscle damage; and increased exercise capacity.

3. The method according to claim 1, wherein the totox value of the marine oil is below 10.

4. The method according to claim 1, wherein the marine oil is fish oil.

5. The method according to claim 1, wherein the marine oil content is about 0.5 to 15% by weight based on the total weight of the composition.

6. The method according to claim 1, wherein the content of the juice is about 20-97% by weight based on the total weight of the composition.

7. The method according to claim 6, wherein said juice is selected from fruit or berries having a high level of antioxidants.

8. The method according to claim 6, herein the juice is selected from the group consisting of: pomegranate, apricot, grapefruit, orange, cranberry, rosehip, pineapple, black chokeberry, mulberry, cloudberry, acerola, raspberry, watermelon, peach, grapes, cherry, jambolao, apple, mango, pear, aronia, passion fruit, beet root, carrot and kiwi.

9. The method according to claim 1, wherein said emulsifier is selected from the group consisting of: milk solids, whey protein, oat protein and pea protein.

10. The method according to claim 1, wherein the composition further comprises pectin.

11. The method according to claim 1, wherein the composition further comprises a sweetener, a flavoring agent, an antioxidant and a preservative.

12. The method according to claim 1, wherein the composition is administered such that the dosage of each of eicosapentaenoic acid and docosahexaenoic acid is in the range from about 200 mg/day to about 5000 mg/day.
